(19) United States
(12) Patent Application Publication
(10) Pub. No.: US 2003/0192131 A1
(43) Pub. Date: Oct. 16, 2003

(54) COMPOSITION FOR THE OXIDATION DYEING OF KERATIN FIBRES AND DYEING PROCESS USING THIS COMPOSITION

(76) Inventor: MARIE-PASCALE AUDOUSSET, ASNIERES (FR)

Correspondence Address: FINNEGAN HENDERSON FARABOW, GARRETT & DUNNER LLP, 1300 I STREET NW, WASHINGTON, DC, 200053315

(21) Appl. No.: 09/443,505

(22) Filed: Nov. 19, 1999

(30) Foreign Application Priority Data
Nov. 20, 1998 (FR) 98 14654

(51) Int. Cl. A61K 7/13

(52) U.S. Cl. 8/405; 8/407; 8/408; 8/409; 8/411; 8/416; 8/421

(57) ABSTRACT

The invention relates to a composition for the oxidation dyeing of keratin fibers, and in particular human keratin fibers such as the hair, comprising, in a medium which is suitable for dyeing, at least one substituted para-aminophenol as oxidation base, and/or 1,3-bis(β-hydroxyethyl)amino-2-methylbenzene and/or at least one of the addition salts thereof with an acid, as coupler, as well as to the dyeing process using this composition.

The invention relates to a composition for the oxidation dyeing of keratin fibers, and in particular human keratin fibers such as the hair, comprising, in a medium which is suitable for dyeing, at least one substituted para-aminophenol as oxidation base, and/or 1,3-bis(β-hydroxyethyl)amino-2-methylbenzene and/or at least one of the addition salts thereof with an acid, as coupler, as well as to the dyeing process using this composition.

It is known practice to dye keratin fibers, and in particular human hair, with dye compositions containing oxidation dye precursors, in particular ortho- or para-phenylenediamines, ortho- or para-aminophenols and heterocyclic bases, which are generally referred to as oxidation bases. Oxidation dye precursors, or oxidation bases, are colourless or weakly coloured compounds which, when combined with oxidizing products, can give rise to coloured compounds and dyes by a process of oxidative condensation.

It is also known that the shades obtained with these oxidation bases can be varied by combining them with couplers or coloration modifiers, the latter being chosen in particular from aromatic meta-diamines, meta-aminophenols, meta-diphenols and certain heterocyclic compounds.

The variety of molecules used as oxidation bases and couplers makes it possible to obtain a wide variety of colours.

The so-called “permanent” coloration obtained by means of these oxidation dyes must moreover satisfy a certain number of requirements. Thus, it must allow shades to be obtained in the desired intensity and it must show good fastness with respect to external agents (light, bad weather, washing, permanent waving, perspiration, rubbing).

The dyes must also make it possible to cover white hairs and, lastly, they must be as unselective as possible, i.e. they must give the smallest possible differences in coloration along the length of the same keratin fiber, which may in fact be differently sensitized (i.e. damaged) between its tip and its root.

Compositions for the oxidation dyeing of keratin fibers containing one or more 2,6-diaminotoluene derivatives as coupler, in combination with one or more oxidation bases which may be chosen from the oxidation bases conventionally used in the field of oxidation dyeing, such as, for example, unsubstituted para-aminophenol and para-phenylenediamine, have already been proposed, in particular in patent application DE-A-4,132,615.

Compositions for the oxidation dyeing of keratin fibers containing one or more 2,6-diaminotoluene derivatives as coupler, in combination with one or more oxidation bases selected from pyrimidine oxidation bases and 2-β-hydroxyethyl-para-phenylenediamine, where these compositions may also contain para-aminophenol derivatives such as 3-methyl-para-aminophenol and 2-methyl-para-aminophenol as additional oxidation base, have also already been proposed, in particular in patent application DE-A-1 9,637,371.

However, although the colorations obtained using such compositions are highly chromatic, they are not entirely satisfactory, in particular as regards their fastness with respect to various treatments and attacking factors to which keratin fibers may be subjected.

The Applicant has just discovered that it is possible to obtain novel dyes which are capable of giving intense, highly chromatic colorations which show excellent fastness with respect to the various attacking factors to which the fibers may be subjected, by combining at least one oxidation base selected from para-aminophenol derivatives of formula (I) defined below, and 1,3-bis(β-hydroxyethyl)amino-2-methylbenzene and/or at least one of the addition salts thereof with an acid as coupler, the said composition being free of any oxidation base which would be chosen from pyrimidine and derivatives thereof, 2-β-hydroxyethyl-para-phenylenediamine and the addition salts thereof with an acid.

This discovery forms the basis of the present invention.

A first subject of the invention is thus a composition for the oxidation dyeing of keratin fibers, and in particular human keratin fibers such as the hair, characterized in that it comprises, in a medium which is suitable for dyeing:

at least one oxidation base chosen from the substituted para-aminophenols of formula (I) below, and the addition salts thereof with an acid:

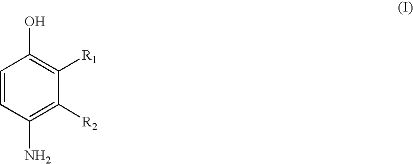

in which:

R₁ represents a hydrogen or fluorine atom, a C₁-C₄ alkyl radical, a C₁-C₄ monohydroxyalkyl radical, a C₂-C₄ polyhydroxyalkyl radical, a C₁-C₄ alkoxyalkyl radical, a C₁-C₄ aminoalkyl radical or a monohydroxy(C₁-C₄)alkylamino(C₁-C₄)alkyl radical;

R₂ represents a hydrogen or fluorine atom, a C₁-C₄ alkyl radical, a C₁-C₄ monohydroxyalkyl radical, a C₂-C₄ polyhydroxyalkyl radical, a C₁-C₄ aminoalkyl radical, a cyano(C₁-C₄)alkyl radical or a C₁-C₄ alkoxyalkyl radical, with the proviso that at least one, and only one, of the radicals R₁ and R₂ represents a hydrogen atom;

1,3-bis(β-hydroxyethyl)amino-2-methylbenzene and/or at least one of the addition salts thereof with an acid, as coupler;

the said composition being free of any additional oxidation base chosen from pyrimidine and derivatives thereof, 2-β-hydroxyethyl-para-phenylenediamine, and the addition salts thereof with an acid.

The dye composition in accordance with the invention gives intense, highly chromatic colorations which show excellent fastness properties with respect both to atmospheric agents such as light and bad weather, and to perspiration and the various treatments to which the hair may be subjected.

A subject of the invention is also a process for the oxidation dyeing of keratin fibers using this dye composition.

Among the substituted para-aminophenols of formula (I) above, mention may be made more particularly of 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

The substituted para-aminophenol(s) of formula (I) preferably represent(s) from 0.0005 to 12% by weight approximately relative to the total weight of the dye composition in accordance with the invention, and even more preferably from 0.005 to 6% by weight approximately relative to this weight.

1,3-Bis(β-hydroxyethyl)amino-2-methylbenzene and/or the addition salt(s) thereof with an acid, which are used as coupler according to the invention, preferably represent(s) from 0.001 to 10% by weight approximately relative to the total weight of the dye composition and even more preferably from 0.01 to 5% by weight approximately relative to this weight.

The dye composition in accordance with the invention can also contain one or more additional couplers other than 1,3-bis(β-hydroxyethyl)amino-2-methylbenzene and other than the addition salts thereof with an acid, and/or one or more direct dyes, in particular in order to modify the shades or to enrich them with glints.

Among the couplers which may additionally be present in the dye composition in accordance with the invention, mention may be made in particular of meta-aminophenols, meta-phenylenediamines, meta-diphenols and heterocyclic couplers, and the addition salts thereof with an acid.

When they are present, these additional couplers preferably represent from 0.0001 to 10% by weight approximately relative to the total weight of the dye composition and even more preferably from 0.005 to 5% by weight approximately relative to this weight.

In addition to the substituted para-aminophenol(s) of formula (I), the dye composition in accordance with the invention can also contain one or more additional oxidation bases chosen from the oxidation bases conventionally used for the oxidation dyeing of keratin fibers, and among which mention may be made of para-phenylenediamines with the exclusion of 2-β-hydroxyethyl-para-phenylenediamine and the addition salts thereof with an acid, ortho-phenylenediamines, double bases and non-pyrimidine heterocyclic bases.

When they are present, the additional oxidation base(s) preferably represent(s) from 0.0005 to 12% by weight approximately relative to the total weight of the dye composition in accordance with the invention, and even more preferably from 0.005 to 6% by weight approximately relative to this weight.

In general, the addition salts with an acid which can be used in the context of the dye compositions of the invention (oxidation bases and couplers) are chosen in particular from the hydrochlorides, hydrobromides, sulphates, tartrates, lactates and acetates.

The medium which is suitable for dyeing (or support) for the dye composition in accordance with the invention generally consists of water or of a mixture of water and at least one organic solvent to dissolve the compounds which would not be sufficiently soluble in water. Organic solvents which may be mentioned, for example, are C₁-C₄ alkanols such as ethanol and isopropanol.

The solvents can be present in proportions preferably of between 1 and 40% by weight approximately relative to the total weight of the dye composition, and even more preferably between 5 and 30% by weight approximately.

The pH of the dye composition in accordance with the invention is generally between 3 and 12 approximately, and preferably between 5 and 12 approximately. It can be adjusted to the desired value by means of acidifying or basifying agents usually used for dyeing keratin fibers.

Among the acidying agents which may be mentioned, for example, are inorganic or organic acids, such as hydrochloric acid, orthophosphoric acid, sulphuric acid, carboxylic acids such as acetic acid, tartaric acid, citric acid and lactic acid, and sulphonic acids.

Among the basifying agents which may be mentioned, for example, are aqueous ammonia, alkaline carbonates, alkanolamines such as mono-, di- and triethanolamine, 2-methyl-2-aminopropanol and derivatives thereof, sodium hydroxide, potassium hydroxide and the compounds of formula (II) below:

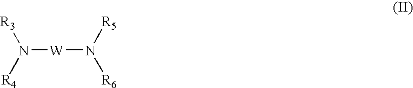

in which W is a propylene residue optionally substituted with a hydroxyl group or a C₁-C₄ alkyl radical; R₃, R₄, R₅ and R₆, which may be identical or different, represent a hydrogen atom, a C₁-C₄ alkyl radical or a C₁-C₄ hydroxyalkyl radical.

The dye composition in accordance with the invention can also contain various adjuvants conventionally used in compositions for dyeing the hair.

Needless to say, a person skilled in the art will take care to select this or these additional optional compounds such that the advantageous properties intrinsically associated with the dye composition in accordance with the invention are not, or are not substantially, adversely affected by the addition(s) envisaged.

The dye composition in accordance with the invention can be in various forms, such as in the form of liquids, creams or gels, which may be pressurized, or in any other form which is suitable for dyeing keratin fibers, and in particular human hair.

A subject of the invention is also a process for dyeing keratin fibers, and in particular human keratin fibers such as the hair, using the dye composition as defined above.

According to this process, the dye composition as defined above is applied to the fibers, the colour being revealed at acidic, neutral or alkaline pH with the aid of an oxidizing agent which is added to the dye composition only at the time of use, or which is present in an oxidizing composition that is applied simultaneously or sequentially in a separate manner.

According to one particularly preferred embodiment of the dyeing process according to the invention, the dye composition described above is mixed, at the time of use, with an oxidizing composition containing, in a medium which is suitable for dyeing, at least one oxidizing agent present in an amount which is sufficient to develop a coloration. The mixture obtained is then applied to the keratin fibers and is left to stand on the fibers for 3 to 50 minutes approximately, preferably 5 to 30 minutes approximately, after which the fibers are rinsed, washed with shampoo, rinsed again and dried.

The oxidizing agent present in the oxidizing composition as defined above may be chosen from the oxidizing agents conventionally used for the oxidation dyeing of keratin fibers, and among which mention may be made of hydrogen peroxide, urea peroxide, alkali metal bromates, persalts such as perborates and persulphates, peracids, enzymes such as 2-electron oxidoreductases, peroxidases and lactases. Hydrogen peroxide is particularly preferred.

The pH of the oxidizing composition containing the oxidizing agent as defined above is such that, after mixing with the dye composition, the pH of the resulting composition applied to the keratin fibers preferably ranges between 3 and 12 approximately and even more preferably between 5 and 11. It is adjusted to the desired value with the aid of acidifying or basifying agents usually used for dyeing keratin fibers and as defined above.

The oxidizing composition as defined above can also contain various adjuvants used conventionally in compositions for dyeing the hair and as defined above.

The composition which is finally applied to the keratin fibers can be in various forms, such as in the form of liquids, creams or gels, or in any other form which is suitable for dyeing keratin fibers, and in particular human hair.

Another subject of the invention is a multi-compartment dyeing device or “kit” or any other multi-compartment packaging system, a first compartment of which contains the dye composition as defined above and a second compartment of which contains the oxidizing composition as defined above. These devices can be equipped with a means for delivering the desired mixture onto the hair, such as the devices described in patent FR-2,586,913 in the name of the Applicant.

The examples which follow are intended to illustrate the invention without thereby limiting its scope.

### EXAMPLES

### Examples 1 and 2 of Dyeing in Alkaline Medium

| EXAMPLE | 1 | | 2 | |
|---|---|---|---|---|
| 3-Methyl-4-aminophenol (oxidation base of | 0.49 | | — | |
| formula (I)) | | | | |
| 4-Amino-2-(β-hydroxyethylaminomethyl)phenol | — | | 1.0 | |
| dihydrochloride (oxidation base of formula (I)) | | | | |
| 1,3-Bis(β-hydroxyethyl)amino-2-methylbenzene | 0.84 | | 0.84 | |
| (coupler) | | | | |
| Common dye support | (*) | | (*) | |
| Demineralized water qs | 100 | g | 100 | g |

(*): Common dye support
96° Ethanol 18 g
Sodium metabisulphite as an aqueous 35% solution 0.68 g
Pentasodium salt of diethylenetriamine pentaacetic acid 1.1 g
Aqueous ammonia containing 20% NH₃ 10 g

Each of the dye compositions described above was mixed, at the time of use, with an equivalent amount by weight of 20-volumes hydrogen peroxide (6% by weight) with a pH of about 3.

Each resulting mixture had a pH of about 10±0.2, and was applied for 30 minutes to locks of permanent-waved grey hair containing 90% white hairs.

The hair was then rinsed with water, washed with a standard shampoo, rinsed again and then dried.

The hair was dyed in the shades given in the table below:

| EXAMPLE | SHADE OBTAINED |
|---|---|
| 1 | Pink |
| 2 | Chromatic dark purple |

### Comparative Examples 3 to 5

The dye compositions below were prepared (contents in grams):

| EXAMPLE | 3(**) | | 4 | | 5 | |
|---|---|---|---|---|---|---|
| para-Aminophenol (oxidation base | 0.327 | | — | | — | |
| not forming part of the invention) | | | | | | |
| 3-Methyl-4-aminophenol (oxidation | | | 0.369 | | — | |
| base of formula (I)) | | | | | | |
| 4-Amino-3-fluorophenol (oxidation | | | — | | 0.381 | |
| base of formula (I)) | | | | | | |
| 1,3-Bis(β-hydroxyethyl)amino-2- | 0.63 | | 0.63 | | 0.63 | |
| methylbenzene (coupler) | | | | | | |
| Common dye support No. 2 | (***) | | (***) | | (***) | |
| Demineralized water qs | 100 | g | 100 | g | 100 | g |

(**): Comparative example not forming part of the invention.
(***): Common dye support No. 2:
Oleyl alcohol polyglycerolated with 2 mol of glycerol 4.0 g
Oleyl alcohol polyglycerolated with 4 mol of glycerol, containing 78% active material (A.M.) 5.69 g A.M.
Oleic acid 3.0 g
Oleylamine containing 2 mol of ethylene oxide, sold under the trade name Ethomeen O12 ® by the company Akzo 7.0 g
Diethylaminopropyl laurylaminosuccinamate, sodium salt, containing 55% A.M. 3.0 g A.M.
Oleyl alcohol 5.0 g
Oleic acid diethanolamide 12.0 g
Propylene glycol 3.5 g
Ethyl alcohol 7.0 g
Dipropylene glycol 0.5 g
Propylene glycol monomethyl ether 9.0 g
Sodium metabisulphite as an aqueous solution containing 35% A.M. 0.455 g A.M.
Ammonium acetate 0.8 g
Antioxidant, sequestering agent qs
Fragrance, preserving agent qs
Aqueous ammonia containing 20% NH₃ 10.0 g

Each of the dye compositions described above was mixed, at the time of use, with an equivalent amount by weight of 20-volumes hydrogen peroxide (6% by weight) with a pH of about 3.

Each resulting mixture had a pH of about 10±0.2, and was applied for 30 minutes to locks of bleached chestnut-coloured hair.

The hair was then rinsed with water, washed with a standard shampoo, rinsed again and then dried.

A test of resistance to 6 consecutive shampoo washings was carried out on the locks of hair thus dyed with the compositions of Examples 3 to 5 above.

The colour of the locks of dyed hair was evaluated in the Munsell system using a Minolta CM 2002 colorimeter, before the test of resistance to shampooing.

According to the Munsell notation, a colour is defined by the expression HV/C in which the three parameters respectively denote the shade or Hue (H), the intensity or Value (V) and the purity or Chromaticity (C), the oblique line in this expression simply being a convention and not indicating a ratio.

The locks of dyed hair were then subjected to a test of resistance to shampooing. The shampoo washes were carried out in a STAR machine (Système de Traitement Automatisé et Robotique [Automated Robotic Processing System]), which automatically links the washing, rinsing and drying operations in sequence, and as described, for example, in patent applications FR-A-2,751,720 and FR-A-2,752,460.

To do this, each of lock of hair was rolled up in a goblet, the base of which was provided with a buffer in mousse form. Each lock of hair was subjected 6 times to the cycle below:

moistening with water for 1 minute;

drying for 30 seconds;

shampooing for 2 minutes 30 seconds (with DOP® shampoo with camomile, diluted to 2%);

rinsing with water at 33° C. for 2 minutes;

drying for 25 minutes.

The dyed locks were thus subjected to 6 consecutive shampooing tests.

The colour of the locks was then evaluated again in the Munsell system using a Minolta CM 2002 colorimeter.

The difference between the colour of the lock before the shampoo washes and the colour of the lock after the shampoo washes was calculated by applying the Nickerson formula:

ΔE=0.4C₀ΔH+6ΔV+3ΔC

as described, for example, in “Couleur, Industrie et Technique”; pages 14-17; vol. No. 5; 1978.

In this formula, ΔE represents the difference in colour between two locks, ΔH, ΔV and ΔC represent the variation in the absolute value of the parameters H, V and C, and C₀ represents the purity of the lock relative to which it is desired to evaluate the colour difference, i.e. the purity of the lock before the shampooing-fastness test.

The degradation of the colour (ΔE) is proportionately greater the higher the value indicated.

The results obtained are given in the table below:

| | | Colour of the hair | | | | |
|---|---|---|---|---|---|---|
| | Colour of the hair | after | Degradation of the | | | |
| EXAM- | before the | the shampoo | colour | | | |
| PLE | shampoo washes | washes | ΔH | ΔV | ΔC | ΔE |
| 3(**) | 0.6 R 3.1/5.8 | 7.8 R 4.1/4.1 | 7.2 | 1.0 | 1.7 | 27.8 |
| 4 | 5.9 R 3.9/5.1 | 0.8 YR 4.5/4.2 | 4.9 | 0.6 | 0.9 | 16.3 |
| 5 | 4.4 R 3.8/5.0 | 0.3 YR 4.3/4.1 | 5.9 | 0.5 | 0.9 | 17.5 |

(**)Comparative example not forming part of the invention.

These results show that the composition of Example 3 not forming part of the invention and as described, for example, in patent application DE 4,132,615, containing a combination of 1,3-bis(β-hydroxyethyl)amino-2-methylbenzene as coupler and para-aminophenol as oxidation base, gives a coloration which is much less fast with respect to the action of shampooing than the colorations obtained using the compositions of Example 4 or 5 in accordance with the invention, i.e. compositions containing a combination of the same coupler but with a substituted para-aminophenol.

What is claimed is:

1. A composition for the oxidation dyeing of keratin fibers comprising, in a medium which is suitable for dyeing:
(a) at least one oxidation base chosen from a substituted para-aminophenol corresponding to formula (I), and an addition salt thereof with an acid: 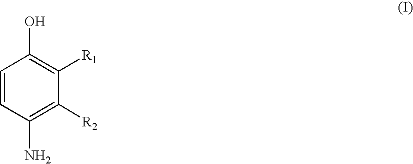
in which:
R₁ is chosen from hydrogen, fluorine, C₁-C₄ alkyl radicals, C₁-C₄ monohydroxyalkyl radicals, C₂-C₄ polyhydroxyalkyl radicals, C₁-C₄ alkoxyalkyl radicals, C₁-C₄ aminoalkyl radicals, and monohydroxy(C₁-C₄)alkylamino(C₁-C₄)alkyl radicals; R₂ is chosen from hydrogen, fluorine, C₁-C₄ alkyl radicals, C₁-C₄ monohydroxyalkyl radicals, C₂-C₄ polyhydroxyalkyl radicals, C₁-C₄ aminoalkyl radicals, cyano(C₁-C₄)alkyl radicals, and C₁-C₄ alkoxyalkyl radicals; and
provided that at least one, and only one, of R₁ and R₂ is hydrogen; and
(b) a coupler chosen from 1,3-bis(β-hydroxyethyl)amino-2-methylbenzene and an addition salt thereof with an acid;
wherein said composition does not include an oxidation base chosen from pyrimidine, pyrimidine derivatives, 2-β-hydroxyethyl-para-phenylenediamine, and addition salt thereof with an acid.

2. The composition according to claim 1, wherein the keratin fibers are human keratin fibers.

3. The composition according to claim 2, wherein the human keratin fibers are hair.

4. The composition according to claim 1, wherein the oxidation base is chosen from 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and an addition salt thereof with an acid.

5. The composition according to claim 1, wherein the oxidation base is present in said composition in an amount ranging from 0.0005 to 12% by weight relative to the total weight of the composition.

6. The composition according to claim 5, wherein the oxidation base is present in said composition in an amount ranging from 0.005 to 6% by weight relative to the total weight of the composition.

7. The composition according to claim 1, wherein the coupler is present in said composition in an amount ranging from 0.001 to 10% by weight relative to the total weight of the composition.

8. The composition according to claim 7, wherein the coupler is present in said composition in an amount ranging from 0.01 to 5% by weight relative to the total weight of the dye composition.

9. The composition according to claim 1, further comprising at least one additional coupler other than 1,3-bis(β-hydroxyethyl)amino-2-methylbenzene and an addition salt thereof with an acid.

10. The composition according to claim 9, further comprising at least one direct dye.

11. The composition according to claim 1, further comprising at least one direct dye.

12. The composition according to claim 1, wherein the addition salt with an acid is chosen from a hydrochloride, a hydrobromide, a sulphate, a tartrate, a lactate, and an acetate.

13. A process for dyeing keratin fibers comprising the steps of 1) applying to said fibers at least one dye composition, and 2) developing a color at acidic, neutral or alkaline pH with the aid of an oxidizing agent which is added to the dye composition only at the time of use, or which is present in an oxidizing composition that is applied simultaneously with the dye composition or sequentially after application of the dye composition, said at least one dye composition comprising, in a medium which is suitable for dyeing:
(a) at least one oxidation base chosen from a substituted para-aminophenol corresponding to formula (I), and an addition salt thereof with an acid: 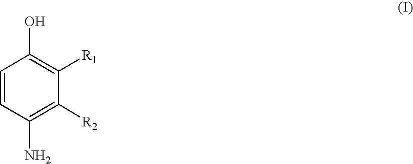
in which:
R₁ is chosen from hydrogen, fluorine, C₁-C₄ alkyl radicals, C₁-C₄ monohydroxyalkyl radicals, C₂-C₄ polyhydroxyalkyl radicals, C₁-C₄ alkoxyalkyl radicals, C₁-C₄ aminoalkyl radicals, and monohydroxy(C₁-C₄)alkylamino(C₁-C₄)alkyl radicals;
R₂ is chosen from hydrogen, fluorine, C₁-C₄ alkyl radicals, C₁-C₄ monohydroxyalkyl radicals, C₂-C₄ polyhydroxyalkyl radicals, C₁-C₄ aminoalkyl radicals, cyano(C₁-C₄)alkyl radicals, and C₁-C₄ alkoxyalkyl radicals;
provided that at least one, and only one, of R₁ and R₂ is hydrogen; and
(b) a coupler chosen from 1,3-bis(β-hydroxyethyl)amino-2-methylbenzene and an addition salt thereof with an acid;
wherein said composition does not include an oxidation base chosen from pyrimidine, pyrimidine derivatives, 2-β-hydroxyethyl-para-phenylenediamine, and an addition salt thereof with an acid.

14. The process according to claim 13, wherein the keratin fibers are human keratin fibers.

15. The process according to claim 14, wherein the human keratin fibers are hair.

16. The process according to claim 13, wherein the oxidizing agent present in the oxidizing composition is chosen from hydrogen peroxide, urea peroxide, alkali metal bromates, persalts, peracids, and enzymes.

17. The process according to claim 16, wherein the persalts are chosen from perborates, percarbonates and persulphates.

18. A multi-compartment dyeing kit comprising a first compartment that contains a dye composition for the oxidation dyeing of keratin fibers comprising, in a medium which is suitable for dyeing:
(a) at least one oxidation base chosen from a substituted para-aminophenol corresponding to formula (I), and an addition salt thereof with an acid: 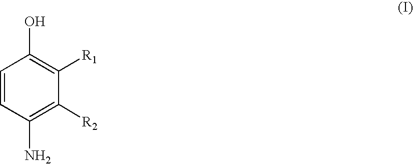
in which:
R₁ is chosen from hydrogen, fluorine, C₁-C₄ alkyl radicals, C₁-C₄ monohydroxyalkyl radicals, C₂-C₄ polyhydroxyalkyl radicals, C₁-C₄ alkoxyalkyl radicals, C₁-C₄ aminoalkyl radicals, and monohydroxy(C₁-C₄)alkylamino(C₁-C₄)alkyl radicals;
R₂ is chosen from hydrogen, fluorine, C₁-C₄ alkyl radicals, C₁-C₄ monohydroxyalkyl radicals, C₂-C₄ polyhydroxyalkyl radicals, C₁-C₄ aminoalkyl radicals, cyano(C₁-C₄)alkyl radicals, and C₁-C4 alkoxyalkyl radicals;
provided that at least one, and only one, of R₁ and R₂ is hydrogen; and
(b) a coupler chosen from 1,3-bis(β-hydroxyethyl)amino-2-methylbenzene and the addition salts thereof with an acid;
wherein said composition does not include an oxidation base chosen from pyrimidine, pyrimidine derivatives, 2-β-hydroxyethyl-para-phenylenediamine, and an addition salt thereof with an acid, and
a second compartment that contains an oxidizing composition.
